(19) United States
(12) Patent Application Publication
(10) Pub. No.: US 2026/0306041 A1
(43) Pub. Date: Oct. 8, 2026

(54) EVALUATION OF CURRENT STATE OF CORONARY ARTERIES USING MACHINE LEARNING

(71) Applicant: Medtronic Vascular, Inc., Santa Rosa, CA (US)

(72) Inventors: Paul J. Coates, Corte Madera, CA (US); Eoghan D. Twohig, Wicklow Town (IE); Binit Panda, Maple Grove, MN (US)

(21) Appl. No.: 19/479,183

(22) PCT Filed: Apr. 18, 2024
(86) PCT No.: PCT/US2024/025186

§ 371 Date: Oct. 27, 2025

Related U.S. Application Data
(60) Provisional application No. 63/500,102, filed on May 4, 2023.

(51) Int. Cl. A61B 6/00; A61B 6/03; A61B 6/12; A61B 6/46; A61B 6/50

(52) U.S. Cl. CPC A61B 6/5217; A61B 6/032; A61B 6/12; A61B 6/461; A61B 6/487; A61B 6/504

(57) ABSTRACT

Example medical systems and techniques are disclosed. An example medical system includes processing circuitry communicatively coupled to memory, the processing circuitry being configured to obtain image data of cardiac anatomy of a patient. The processing circuitry is configured to generate a 3D image of the cardiac anatomy of the patient based on the image data. The processing circuitry is configured to determine an identification of an implanted medical device based on the 3D image. The processing circuitry is configured to determine a recommended treatment strategy based on the 3D image and the identification of the implanted medical device and output the recommended treatment strategy.

This application claims the benefit of priority from U.S. Provisional Patent Application No. 63/500,102, filed May 4, 2023, the entire contents of which are incorporated herein by reference.

### TECHNICAL FIELD

This disclosure relates to the determination of a recommended treatment strategy for a medical procedure.

### BACKGROUND

During a medical procedure, a clinician may use an imaging system to be able to visualize internal anatomy of a patient. Such an imaging system may display anatomy, medical instruments, or the like, and may be used to diagnose a patient condition or assist in guiding a clinician in moving a medical instrument to an intended location inside the patient. Imaging systems may use sensors to capture image data which may be displayed during the medical procedure. Imaging systems include fluoroscopic systems (e.g., isocentric C-arm fluoroscopic systems), intravascular ultrasound (IVUS) systems, other ultrasound imaging systems, computed tomography (CT) scan systems, optical coherence tomography (OCT) fractional flow reserve (FFR) systems, magnetic resonance imaging (MRI) systems, positron emission tomography (PET) systems, as well as other imaging systems. A clinician may determine a treatment strategy based on images captured by such imaging systems.

### SUMMARY

Emergency medical procedures may occur without planning and a clinician performing the emergency medical procedure may be unable to access a patient medical history for the patient of the emergency medical procedure. As such, determining an appropriate or preferred treatment strategy for the emergency medical procedure may be more difficult than if a full patient medical history were available, as prior medical procedures may inform or impact a current medical procedure, such as an emergency medical procedure. As such, it may be desirable to determine a recommended treatment strategy that takes into account prior medical procedure(s) that may impact a current medical procedure even when patient medical history is not available.

In general, this disclosure is directed to various techniques and medical systems for determining and outputting a recommended treatment strategy during or prior to a medical procedure. The recommended treatment strategy may be based on a 3-dimensional (3D) image created from imaging data captured by an imaging system. The 3D image may include an implanted medical device which may either itself impact the current medical procedure or provide insight into previous medical procedure(s) that may impact the current medical procedure. The system and/or techniques may identify the implanted medical device and determine the recommended treatment strategy based on the 3D image and the identified implanted medical device. The system and/or techniques may output and/or display the recommended treatment strategy to and/or for the clinician, who may use the recommended treatment strategy to guide the medical procedure.

In one example, the disclosure describes a medical system comprising memory configured to store a 3-dimensional (3D) image of cardiac anatomy of a patient; and processing circuitry communicatively coupled to the memory, the processing circuitry being configured to: obtain image data of the cardiac anatomy of the patient; generate the 3D image of the cardiac anatomy of the patient based on the image data; determine an identification of an implanted medical device based on the 3D image; determine a recommended treatment strategy based on the 3D image and the identification of the implanted medical device; and output the recommended treatment strategy.

In another example, the disclosure describes a method comprising obtaining, by a medical system, image data of cardiac anatomy of a patient; generating, by the medical system, a 3D image of the cardiac anatomy of the patient based on the image data; determining an identification of an implanted medical device based on the 3D image; determining a recommended treatment strategy based on the 3D image and the identification of the implanted medical device; and outputting the recommended treatment strategy.

In yet another example, the disclosure describes a non-transitory computer readable medium comprising instructions, which, when executed, cause processing circuitry to obtain image data of cardiac anatomy of a patient; generate a 3D image of the cardiac anatomy of the patient based on the image data; determine an identification of an implanted medical device based on the 3D image; determine a recommended treatment strategy based on the 3D image and the identification of the implanted medical device; and output the recommended treatment strategy.

These and other aspects of the present disclosure will be apparent from the detailed description below. In no event, however, should the above summaries be construed as limitations on the claimed subject matter, which subject matter is defined solely by the attached claims.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIG. 1 is a schematic perspective view of one example of a system for determining a recommended treatment strategy according to one or more aspects of this disclosure.

FIG. 2 is a schematic view of one example of a computing system of the system of FIG. 1.

FIG. 3 is a flow diagram of example techniques for determining a recommended treatment strategy according to one or more aspects of this disclosure.

FIG. 4 is a flow diagram illustrating example database generation techniques according to one or more aspects of this disclosure.

FIGS. 5A and 5B are flow diagrams illustrating example machine learning model training techniques according to one or more aspects of this disclosure.

FIG. 6 is a flow diagram illustrating example machine learning model verification techniques according to one or more aspects of this disclosure.

FIG. 7 is a conceptual diagram illustrating an example machine learning model according to one or more aspects of this disclosure.

FIG. 8 is a conceptual diagram illustrating an example training process for a machine learning model according to one or more aspects of this disclosure.

### DETAILED DESCRIPTION

Emergency medical procedures may occur without advanced planning. A patient may suffer from an ailment, such as a cardiac ailment or disease, which may require immediate or timely medical intervention. A clinician performing the emergency medical procedure may not be able to access the patient medical history of the patient undergoing the emergency medical procedure. For example, the emergency medical procedure may be performed at a medical facility that does not have access to the patient medical history because either the medical facility is not previously associated with the patient and/or the patient is not in a condition or state to communicate any previous medical procedures to another. For example, a ST elevated myocardial infarction (STEMI) patient may require an emergency medical procedure to remove an acute vascular blockage, but the STEMI patient is unlikely to be able to communicate their prior coronary medical history to hospital staff.

However, previous medical interventions may complicate a current medical procedure. For example, the STEMI may be a result of in-stent restenosis at a previous stent, a culprit lesion may be distal of a prior stent, or the patient may have had a coronary artery bypass graft (CABG) with artery or vein grafts implanted into the patient, any or all of which may present potential complications for the current medical procedure.

Chronic total occlusions (CTOs) may present an especially difficult problem in that the true lumen of the occluded vessel is “lost” and the lesion blocking the lumen may be treated with an antegrade or a retrograde approach. Identification of a retrograde approach through collateral flow may be difficult given that various vessels may be confused in a 2-dimensional (2D) fluoroscopic image. Clinicians may thus use a trial-and-error approach when attempting to find a desired route to the lesion.

During a STEMI medical procedure, in particular, but potentially during elective coronary interventions, a clinician may be focused on solving a particular and specific urgent problem. During the STEMI medical procedure, the patient anatomy may be visualized, for example, through fluoroscopy, and there may be information in the imaging data that is available to the clinician that may identify other medical issues that may not be identified or noticed by the clinician due to the focus of the clinician on the current intervention rather than diagnosis of other potential issues.

While clinicians do their best during each procedure, not every outcome is positive. A clinician may not have a tool that provides feedback to the physician either during the medical procedure as to how to improve the current device placement, or, to quantify risk or likelihood of future failure (for example, in-stent restenosis).

Imaging systems may be used to assist a clinician during a medical procedure, such as a medical intervention procedure. For example, imaging systems may be used during an emergency medical procedure to guide and assist a clinician with planning and performing various medical intervention tasks. A medical system may use imaging data from such imaging system(s) to determine a 3D image of anatomy of the patient, such as cardiac anatomy. The medical system may use the 3D image to determine an identification of an implanted medical device. The medical system may determine a recommended treatment strategy based on the 3D image and the identification of the implanted medical device. The medical system may output, e.g., for display, and/or display the recommended treatment strategy, which a clinician may use to guide the medical procedure.

Aspects of this disclosure are applicable to at least Catheterization lab (Cath lab) procedures. Example Cath lab procedures include, but are not necessarily limited to, coronary procedures (e.g., STEMI, CTO, CABG, or the like), renal denervation (RDN) procedures, structural heart and aortic (SH&A) procedures (e.g., transcatheter aortic valve replacement (TAVR), transcatheter mitral valve replacement (TMVR), and the like), device implantation procedures (e.g., heart monitors, pacemakers, defibrillators, and the like), etc.

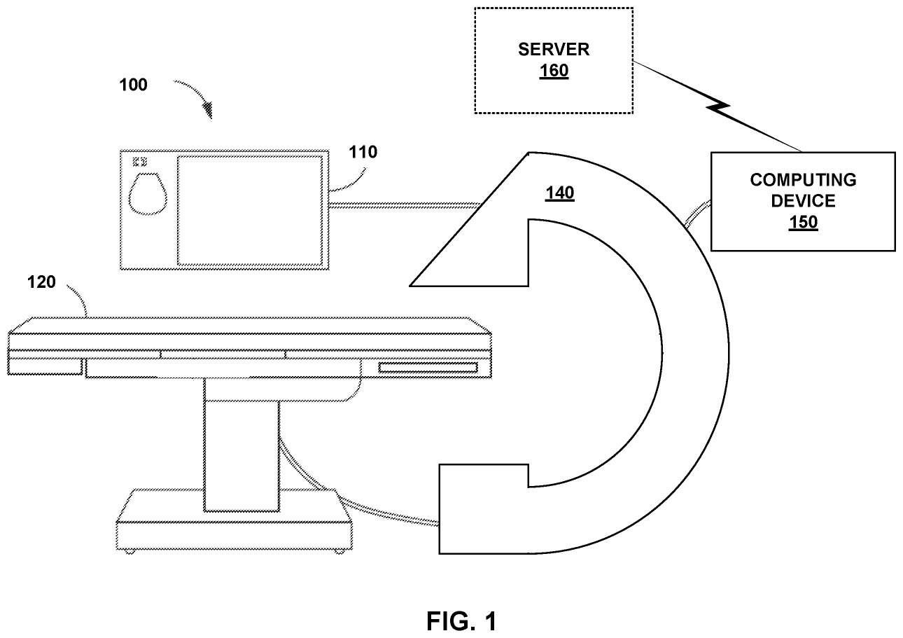

FIG. 1 is a schematic perspective view of one example of a system for determining a recommended treatment strategy according to one or more aspects of this disclosure. System 100 includes a display device 110, a table 120, an imager 140, and a computing device 150. System 100 may be an example of a system for use in an emergency room or a Cath lab. In some examples, system 100 may include other devices, not shown for simplicity purposes. In some examples, system 100 may also include server 160, which may be co-located with the other devices of system 100 or may be located elsewhere. System 100 may be used during a medical procedure, such as an emergency medical procedure to treat a medical condition of a patient. During such a procedure, system 100 may determine and display a recommended treatment strategy, according to the techniques of this disclosure.

System 100 may include one or more machine learning models. A machine learning model may be trained to create a 3D image of anatomy of the patient. A machine learning model may be trained to identify an implanted medical device in the patient, such as a stent, sternal ties, a pacemaker, a ventricular assist device, an insertable cardiac monitor, or other implanted medical device within the patient. For example, sometimes it is difficult for a person to identify whether or not a patient has an implanted stent from viewing image data. A machine learning model may also be trained to determine a recommended treatment strategy based on a 3D image of patient anatomy, such as cardiac anatomy, and the identification of the implanted medical device. For example, a machine learning model may be used to determine a recommended treatment strategy that takes into account prior medical intervention(s) which may be determined to have occurred based on the identification of the implanted medical device. Such a recommended treatment strategy may be a potential treatment having a greatest chance of a successful outcomes given the previous medical intervention(s). In some examples, a machine learning model may provide real-time feedback to a clinician during the medical procedure as to medical device placement, medical instrument placement, risks associated with a given treatment strategy, and/or may update the recommended treatment strategy as more imaging data becomes available.

Computing device 150 may include, for example, an off-the-shelf device such as a laptop computer, desktop computer, tablet computer, smart phone, or other similar device or may include a specific purpose device. Computing device 150 may perform various control functions with respect to imager 140. In some examples, computing device 150 may include a guidance workstation. Computing device 150 may control the operation of imager 140 and receive the output of imager 140.

Display device 110 may be configured to output instructions, images, and messages relating to the medical procedure. Table 120 may be, for example, an operating table or other table suitable for use during a medical procedure.

In the example of FIG. 1, imager 140, such as a fluoroscopy imager, an angiography imager, or other imaging device, may be used to image relevant portions of the patient's anatomy during the medical procedure to visualize the anatomy, characteristics and locations of lesions or other issues inside the patient's body through the generation of imaging data. Imager 140 may also generate imaging data of implanted medical devices within the patient's body, such as stents, sternal ties, or other implanted medical devices. While described primarily as a fluoroscopy imager, imager 140 may be any type of imaging device, such as an angiography device, an intravascular ultrasound IVUS device, an OCT-FFR device, a CT device, an MRI device, a PET device, an ultrasound device, or the like. In some examples, imager 140 may represent more than one imaging device, such as a plurality of any of the aforementioned devices.

Imager 140 may image a region of interest in the patient's body. The particular region of interest may be dependent on anatomy, the medical procedure, patient symptoms, and/or the like. For example, when performing a cardiac medical procedure, a portion of the vasculature and/or the heart may be within the region of interest.

Computing device 150 may be communicatively coupled to imager 140, display device 110 and/or server 160, for example, by wired, optical, or wireless communications. Server 160 may be a hospital server which may or may not be located in an emergency room or Cath lab of a hospital, a cloud-based server, or the like. Server 160 may be configured to store patient imaging data, electronic healthcare or medical records or the like Any of, or any combination of, computing device 150, imager 140, and/or server 160 may include one or more machine learning model(s). For example, computing device 150, imager 140, and/or server 160 may obtain image data of the cardiac anatomy of the patient. Computing device 150, imager 140, and/or server 160 may generate the 3D image of the cardiac anatomy of the patient based on the image data. In some examples, computing device 150, imager 140, and/or server 160 may execute a machine learning model to generate the 3D image. Computing device 150, imager 140, and/or server 160 may determine the identification of an implanted medical device based on the 3D image. In some examples, Computing device 150, imager 140, and/or server 160 may execute a machine learning model to determine the identification of an implanted medical device. Computing device 150, imager 140, and/or server 160, may determine a recommended treatment strategy based on the 3D image and the identification of the implanted medical device. In some examples, computing device 150, imager 140, and/or server 160 may execute a machine learning model to determine a recommended treatment strategy. Computing device 150, imager 140, and/or server 160 may output the recommended treatment strategy, for example, for display to display device 110. For example, a machine learning model may determine the identity of an implanted medical device. In some examples, based on the identity of the implanted medical device and the 3D image, a machine learning model may determine a condition of the implanted medical device and/or previous medical procedure(s) performed on the patient which may cause impediments or potential complications in the current medical procedure. The machine learning model may use such determination(s) to determine a recommended treatment strategy for the current medical procedure.

By providing a recommended treatment strategy that takes into account previous medical procedures, even when the patient's medical records may not be available, the techniques of this disclosure may affect a particular treatment or prophylaxis for a disease or medical condition. These techniques may improve patient outcomes, reduce the need for repeating the medical procedure, speed up the medical procedure, reduce the exposure of the patient to radiopaque contrasts, and/or preserve medical resources.

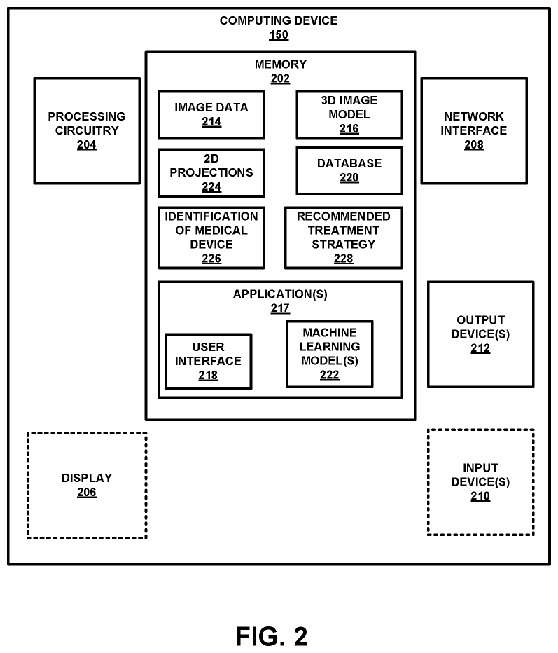

FIG. 2 is a schematic view of one example of a computing device 150 of system 10 of FIG. 1. Computing device 150 may include a workstation, a desktop computer, a laptop computer, a smart phone, a tablet, a dedicated computing device, or any other computing device capable of performing the techniques of this disclosure.

Computing device 150 may be configured to perform processing, control and other functions associated with imager 140. As shown in FIG. 2, computing device 150 may represent multiple instances of computing devices, each of which may be associated with imager 140. Computing device 150 may include, for example, a memory 202, processing circuitry 204, a display 206, a network interface 208, input device(s) 210, and/or output device(s) 212, each of which may represent any of multiple instances of such a device within the computing system, for ease of description.

While processing circuitry 204 appears in computing device 150 in FIG. 2, in some examples, features attributed to processing circuitry 204 may be performed by processing circuitry of any of computing device 150, imager 140, or server 160, or combinations thereof. In some examples, one or more processors associated with processing circuitry 204 in computing system may be distributed and shared across any combination of computing device 150, imager 140, and server 160. Computing device 150 may be used to perform any of the techniques described in this disclosure, and may form all or part of devices or systems configured to perform such techniques, alone or in conjunction with other components, such as components of computing device 150, imager 140, server 160, or a system including any or all of such systems.

Memory 202 of computing device 150 includes any non-transitory computer-readable storage media for storing data or software that is executable by processing circuitry 204 and that controls the operation of computing device 150 and/or imager 140, as applicable. In one or more examples, memory 202 may include one or more solid-state storage devices such as flash memory chips. In one or more examples, memory 202 may include one or more mass storage devices connected to the processing circuitry 204 through a mass storage controller (not shown) and a communications bus (not shown).

Although the description of computer-readable media herein refers to a solid-state storage, it should be appreciated by those skilled in the art that computer-readable storage media may be any available media that may be accessed by the processing circuitry 204. That is, computer readable storage media includes non-transitory, volatile and non-volatile, removable and non-removable media implemented in any method or technology for storage of information such as computer-readable instructions, data structures, program modules, or other data. For example, computer-readable storage media includes RAM, ROM, EPROM, EEPROM, flash memory or other solid state memory technology, CD-ROM, DVD, Blu-Ray or other optical storage, magnetic cassettes, magnetic tape, magnetic disk storage or other magnetic storage devices, or any other medium that may be used to store the desired information and that may be accessed by computing device 150. In one or more examples, computer-readable storage media may be stored in the cloud or remote storage and accessed using any suitable technique or techniques through at least one of a wired or wireless connection.

Memory 202 may store image data 214, 3D image model 216, 2D projections 224, database 220, identification of medical device 226 and/or recommended treatment strategy 228. Image data 214 may be generated by imager 140 of anatomy of the patient and obtained by computing device 150 via network interface 208 which may be communicatively coupled to imager 140. In some examples, image data 214 may include image data indicative of an implanted medical device in the patient, such as a stent, sternal ties, or other implanted medical device. Memory 202 may store 3D image model 216. For example, processing circuitry 204 may generate 3D image model 216 based on image data 214. In some examples, 3D image model 216 may include a 3D model of anatomy of the patient. In some examples, 3D image model 216 may include data indicative of the implanted medical device. 2D projections 224 may include 2D projections of 3D image model 216 which processing circuitry 204 may use to identify an implanted medical device and/or previous medical procedures, or potential complications relating thereto. Database 220 may include a database containing images and/or image features associated with various implantable medical devices and characteristics thereof, such as dimensions (e.g., length, diameter, or the like). In some examples, database 220 may include a stent database which may be used by processing circuitry 204 to identify an implanted stent in the patient.

Image data 214 may be captured by imager 140 (FIG. 1) during a medical procedure of a patient. Processing circuitry 204 may obtain imaging data 214 from imager 140 and store image data 214 in memory 202. Processing circuitry 204 may execute user interface 218 so as to cause display 206 (and/or display device 110 of FIG. 1) to present user interface 218 to one or more clinicians performing the medical procedure. Memory 202 may also store one or more machine learning model(s) 222 and user interface 218. Machine learning model(s) 222 may be configured to, when executed by processing circuitry 204, generate 3D image model 216 based on image data 214. In some examples, image data 214 may represent 2D planes of data and as such, not all image data pertaining to 3D image model 216 may be available from imager 140 imaging the patient. In such examples, processing circuitry 204 executing machine learning model(s) 222 may make predictions of patient anatomy which may be used by processing circuitry 204 to generate 3D image model 216.

In some examples, processing circuitry 204 may execute machine learning model(s) 222 to determine an identification of an implanted medical device based on 3D image model 216 (or image data 214). Machine learning model(s) 222 may also be configured to, when executed by processing circuitry 204, determine a recommended treatment strategy based on 3D image model 216 (or image data 214) and identification of implanted medical device 226.

In some examples, processing circuitry 204 may create 3D image model 216 of cardiac anatomy based on image data 214 (e.g., fluoroscopy data). For example, processing circuitry 204 may use multiplane fluoroscopy image data 214 to generate 3D image model 216. In some examples, 3D image model 216 may include a 3D model of anatomy of the patient. Processing circuitry 204 may review 3D image model 216 and identify the presence of previous interventions, for example, a stent or sternal ties (which may suggest a prior cardiac artery bypass graft (CABG)). In some examples, processing circuitry 204 may execute machine learning model(s) 222 to generate 3D image model 216. In such examples, machine learning model(s) 222 may be trained using image data of “clean” anatomy (e.g., anatomy without previous intervention) and examples of image data of anatomy previously treated (e.g., anatomy with previous intervention).

In some examples, processing circuitry 204 may attempt to determine whether there is an implanted medical device in the patient. For example, processing circuitry 204 may execute machine learning model(s) 222 to review image data 214 and/or 3D image model 216 to determine whether an implanted medical device appears in image data 214 and/or 3D image model 216. Processing circuitry 204 may identify characteristics of the implanted medical device, such as dimensions of the implanted medical device in database 220 to determine an identification of the implanted medical device. Database 220 may be obtained from another device or may be generated by processing circuitry 204 as discussed herein elsewhere. The identification of the implanted medical device may provide insight into possible impediments or complications to the current medical procedure.

In some examples, database 220 may include a stent database having stent information, such as stent length, diameter, design, manufacturer, model, etc. In some examples, processing circuitry 204 may determine that there is a previously implanted stent in the patient. Processing circuitry 204 may attempt to identify the stent using specific features of the stent, such as the length of the stent (e.g., based on stent features, like repeat units) and assess the effectiveness of the stent's deployment. A repeat unit may be a single structure that is repeated a plurality of times in a stent. As such, a nominal length of the stent may depend on a number of repeat units of the stent. Processing circuitry 204 may use such parameters to provide information and/or a recommendation as to the ability to cross the stent (e.g., if a new lesion is distal to the stent), or information and/or a recommendation as to treatment if new lesion is in-stent restenosis (ISR). For example, if a prior stent is to be crossed to treat a current condition, processing circuitry 204 might recommend that the previous stent be addressed, for example, by providing additional dilatation, before attempting to cross the previous stent. For example, processing circuitry 204 might recommend one or more preferred stents that might be best able to cross the lesion, or other tools that might facilitate crossing, for example wire, buddy wire, guide extension catheter selection, etc. In the instance of ISR, processing circuitry 204 may perform an examination of the previous stent (design, length, deployment state, etc.) and may recommend a treatment strategy based on such an examination. For example, processing circuitry 204 may recommend full length dilatation with a non-compliant balloon, spot dilatation, using a drug-eluting balloon (DEB), deploying a new stent, etc. In some examples, processing circuitry 204 may provide an assessment of risk associated with a given recommended treatment strategy (e.g., risky, not risky, an alphanumeric indication of likelihood of success, or the like). In some examples, if the implanted medical device is in poor condition or the placement of the implanted medical device is not optimal, processing circuitry 204 may provide feedback to a clinician regarding the implanted medical device or surrounding tissue. For example, processing circuitry 204 may warn the clinician to watch out for spokes on a distal portion of an implanted stent.

If processing circuitry 204 identifies sternal ties in image data 214 and/or 3D image model 216, there may have been a prior CABG and therefore, it may be likely that one or more native vessel(s) are occluded and bypassed. Processing circuitry 204 may employ image analysis to track contrast flow to find any bypass graft, assess patency, and provide recommendations as to tools appropriate for passage.

In some examples, 3D image model 216 may include a 3D map of coronary arteries. Based on anatomical structure of the 3D map of coronary arteries, processing circuitry 204 may determine the most likely path and diameter of the artery section with a chronic total occlusion (CTO). Processing circuitry 204 may determine collateral flow based on the 3D map and identify if a retrograde approach is practical. If multiple routes are possible, processing circuitry 204 may identify an optimal retrograde path and provide recommendation(s) as to the equipment most likely to make the route practicable (wire choice(s), microcatheter(s), etc.). In some examples, processing circuitry 204 may determine an estimated risk or likelihood of success associated with each of the multiple routes as part of determining recommended treatment strategy 228.

In some examples, processing circuitry 204 may utilize image data 214 to perform additional cardiac evaluation “in the background” during the medical procedure. For example, fluoroscopy may not be the best tool for all aspects of evaluation, but there are certain aspects of cardiac anatomy that can be explored with fluoroscopy. For example, fluoroscopy may be used to determine if calcium is present on the aortic valve, if the left atrial appendage has a shape and/or configuration that might be prone to produce and shed clots, if the heart looks abnormal (which may indicate issues with ventricular or atrial pumping). Such information may be used by processing circuitry 204 to determine potential issues and/or recommended treatment strategies. In some examples, processing circuitry 204 may identify other potential medical issues based on image data 214 which may not be the focus of the current medical procedure. In such examples, processing circuitry 204 may flag such potential issues for follow-up by the clinician, e.g., later during the current procedure and/or during a follow-on procedure in the future. Processing circuitry 204 may, executing machine learning model(s) 222, determine a recommended treatment strategy for treating any potential issues as well.

In some examples, processing circuitry 204 may integrate interventional imaging data (e.g., fluoroscopy, IVUS, OCT-FFR, etc.) and provide feedback to a clinician. For example, processing circuitry 204 may provide recommendations to improve outcomes by offering guidance as to how to modify device placement during the procedure, for example, by identifying under-deployment of a section of a stent and recommending a high-pressure non-compliant balloon deployment. In some examples, processing circuitry 204 may recommend balloon length, device placement based on knowledge of marker band placement, and/or deployment pressure). In some examples, processing circuitry 204 may assesses final state of device placement and provides a risk or risk/reward analysis as to a likelihood of success, or a need to have higher post-procedural follow-up. Processing circuitry 204 may be trained based on prior procedures to support the ability to provide guidance for the current procedure.

In some examples, processing circuitry 204 executing machine learning model(s) 222 may provide real-time feedback to a clinician during the medical procedure as to medical device placement, medical instrument placement, risks associated with a given treatment strategy, and/or may update the recommended treatment strategy as more imaging data becomes available. For example, not every procedure will be taken to best outcome as doing so may involve higher risk. As such, machine learning model(s) 222 may use accumulated knowledge from training to provide a recommendation of when to stop a given portion of a procedure due to a risk/reward balancing. In some examples, processing circuitry 204 may monitor health of the patient via one or more sensors of equipment of system 100 and may generate or modify a recommended treatment strategy further based on one or more sensor signals, for example, representing physiological parameters of the patient.

Processing circuitry 204 may be implemented by one or more processors, which may include any number of fixed-function circuits, programmable circuits, or a combination thereof. In various examples, control of any function by processing circuitry 204 may be implemented directly or in conjunction with any suitable electronic circuitry appropriate for the specified function. Fixed-function circuits refer to circuits that provide particular functionality and are preset on the operations that may be performed. Programmable circuits refer to circuits that may programmed to perform various tasks and provide flexible functionality in the operations that may be performed. For instance, programmable circuits may execute software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. In some examples, the one or more of the units may be distinct circuit blocks (fixed-function or programmable), and in some examples, the one or more units may be integrated circuits.

Instructions may be executed by one or more processors, such as one or more digital signal processors (DSPs), general purpose microprocessors, application specific integrated circuits (ASICs), field programmable gate arrays (FPGAs), graphics processing units (GPUs) or other equivalent integrated or discrete logic circuitry. Accordingly, the term processing circuitry 204 as used herein may refer to one or more processors having any of the foregoing processor or processing structure or any other structure suitable for implementation of the techniques described herein. In addition, in some aspects, the functionality described herein may be provided within dedicated hardware or software modules configured for encoding and decoding, or incorporated in a combined codec. Also, the techniques could be fully implemented in one or more circuits or logic elements.

Display 206 may be touch sensitive or voice activated (e.g., via one or more sensors 230 which may include one or more microphones), enabling display 206 to serve as both an input and output device. Alternatively, a keyboard (not shown), mouse (not shown), or other data input devices (e.g., input device(s) 210) may be employed.

Network interface 208 may be adapted to connect to a network such as a local area network (LAN) that includes a wired network or a wireless network, a wide area network (WAN), a wireless mobile network, a Bluetooth network, or the internet. For example, computing device 150 may obtain image data 214 from imager 140 during a medical procedure. Computing device 150 may receive updates to its software, for example, application(s) 217, via network interface 208. Computing device 150 may also display notifications on display 206 that a software update is available.

Input device(s) 210 may include any device that enables a user to interact with computing device 150, such as, for example, a mouse, keyboard, foot pedal, touch screen, augmented-reality input device receiving inputs such as hand gestures or body movements, or voice interface.

Output device(s) 212 may include any connectivity port or bus, such as, for example, parallel ports, serial ports, universal serial busses (USB), or any other similar connectivity port known to those skilled in the art.

Application(s) 217 may be one or more software programs stored in memory 202 and executed by processing circuitry 204 of computing device 150. Processing circuitry 204 may execute user interface 218, which may display image data 214, representations of 3D image model 216, 2D projections 224, identification of medical device 226, and/or recommended treatment strategy 228 on display 206 and/or display device 110. A clinician may use recommended treatment strategy 228 and other data being displayed to guide the medical procedure. In some examples, recommended treatment strategy 228 may be overlayed on image data 214, representations of 3D image model 216, and/or 2D projections 224 to visually assist the clinician in performing the medical procedure.

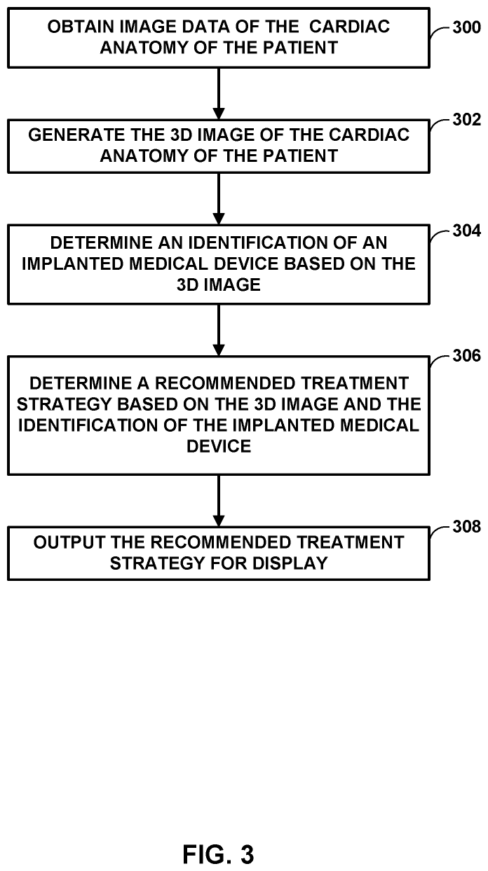

FIG. 3 is a flow diagram of example techniques for determining a recommended treatment strategy according to one or more aspects of this disclosure. The techniques of FIG. 3 are described below with respect to processing circuitry 204, but such techniques may be performed by any of, or any combination of, processing circuitry of devices depicted in FIG. 1 or capable of performing such techniques.

Processing circuitry 204 may obtain image data of the cardiac anatomy of the patient (300). For example, processing circuitry 204 may receive image data 214 from imager 140 via network interface 208. Processing circuitry 204 may generate the 3D image of the cardiac anatomy of the patient based on the image data (302). For example, processing circuitry 204 may generate 3D image model 216 based on image data 214 using any known 3D modeling or image assembly techniques. In some examples, processing circuitry 204 may execute machine learning model(s) 222 as part of generating 3D image model 216.

Processing circuitry 204 may determine an identification of an implanted medical device based on the 3D image (304). For example, processing circuitry 204 may determine the identity of a stent that is implanted in the patient based on characteristics of the stent which may be determined from the 3D image. Processing circuitry 204 may look up those characteristics of the stent in database 220 to determine the identification of the stent. In some examples, processing circuitry 204 may execute machine learning model(s) 222 as part of identifying the implanted medical device.

Processing circuitry 204 may determine a recommended treatment strategy based on the 3D image and the identification of the implanted medical device (306). For example, processing circuitry 204 may execute machine learning model(s) 222 and, based on 3D image model 216 and identification of medical device 226, determine recommended treatment strategy 228. For example, 3D image model 216 may contain information indicative of a current state of the anatomy of the patient. Identification of medical device 226 may provide information about previous medical procedure(s) which may otherwise complicate a current medical procedure. By determining recommended treatment strategy 228 while taking into account previous medical procedure(s), even when patient medical history records are not available, patient outcomes may be improved.

Processing circuitry 204 may output the recommended treatment strategy (308). For example, processing circuitry may control display 206 to display recommended treatment strategy 228 and/or may output, via network interface 208, recommended treatment strategy 228 for display on display device 110 (FIG. 1).

In some examples, part of at least one of generating the 3D image of the cardiac anatomy of the patient based on the image data, determining the identification of the implanted medical device based on the 3D image, or determining the recommended treatment strategy based on the 3D image and the identification of the implanted medical device, processing circuitry 204 may execute at least one machine learning model.

In some examples, as part of determining the identification of the implanted medical device, processing circuitry 204 may determine that at least one characteristic of the implanted medical device matches at least one characteristic of a stent in a stent database (e.g., in database 220). In some examples, the at least one characteristic comprises one or more of a type, a length, or a diameter.

In some examples, processing circuitry 204 may determine an effectiveness of a previous medical procedure based on the 3D image and the identification of the implanted medical device. In some examples, the implanted medical device includes a sternal tie.

In some examples, as part of determining the recommended treatment strategy, processing circuitry 204 may determine a path to an artery of the cardiac anatomy having a chronic total occlusion and a diameter of the artery of the cardiac anatomy having the chronic total occlusion. In some examples, the recommended treatment strategy includes the path to the artery and the diameter of the artery.

In some examples, the recommended treatment strategy includes a recommended retrograde path and at least one recommended equipment. In some examples, the image data comprises at least one of fluoroscopy data, IVUS data, or OCT-FFR data.

In some examples, processing circuitry 204 may obtain a database of implantable medical devices (e.g., database 220). In some examples, processing circuitry 204 may generate the database of implantable medical devices. For example, processing circuitry 204 may create respective 3D models of each of a plurality of implantable medical devices. Processing circuitry 204 may create a plurality of 2D projections of each respective 3D model. Processing circuitry 204 may collate lengths and diameters of the plurality of 2D projections of each respective 3D model. Processing circuitry 204 may define rules for database 220.

In some examples, machine learning model(s) 222 is trained on a plurality of fluoroscopy images of implanted medical devices. In some examples, the plurality of fluoroscopy images includes a first set of fluoroscopy images of implanted medical devices and a second set of fluoroscopy images of implanted medical devices, wherein the second set of fluoroscopy images comprise images of implanted medical devices in less desirable implantation situations than the first set of fluoroscopy images. For example, the first set of fluoroscopy images may include relatively clean or relatively ideal images of relatively properly placed medical devices, while the second set of fluoroscopy images may include relatively unclean or relatively non-ideal images of medical devices, either due to image quality, medical device condition, or medical device placement.

In some examples, the training of machine learning model(s) 222 is verified using a third set of fluoroscopy images of implanted medical devices. For example, a person may use a third set of images that is different from the first and second sets of images as input to machine learning model(s) 222. Machine learning model(s) 222 may classify the third set of images as representing different implanted medical devices. The person may then grade the classification provided by machine learning model(s) 222 in a supervised learning process, for example, by using information from patient medical records, inventory, and/or the third set of images.

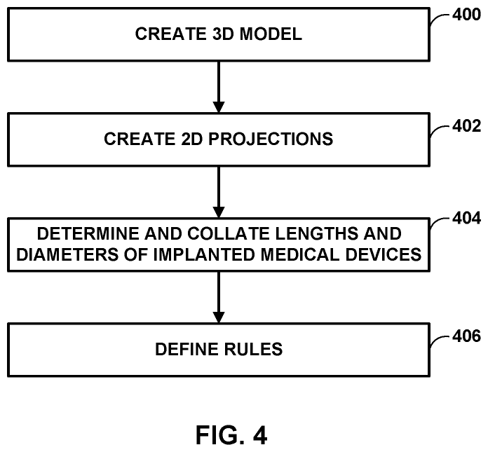

FIG. 4 is a flow diagram illustrating example database generation techniques according to one or more aspects of this disclosure. The techniques of FIG. 4 are described below with respect to processing circuitry 204, but such techniques may be performed by any of, or any combination of, processing circuitry of devices depicted in FIG. 1 or capable of performing such techniques.

Processing circuitry 204 may a create 3D model (e.g., 3D image model 216) (400). Processing circuitry 204 may create 2D projections 224 (402). For example, processing circuitry 204 may create a plurality of 2D projections 224 based on 3D image model 216. 2D projections 224 may represent different 2D planes at different angles of 3D image model 216. From such 2D projections, processing circuitry 204 may determine lengths and diameters of any represented implanted medical devices (404). Processing circuitry 204 may define rules for database 220 (406). For example, processing circuitry 204 may define rules used to create database 220, associations amongst various cells of database 220, searching rules for database 220, or the like. Processing circuitry 204 may generate a hierarchy of rules that machine learning model may follow to determine brand, size, deployment quality of determined medical device based on training data.

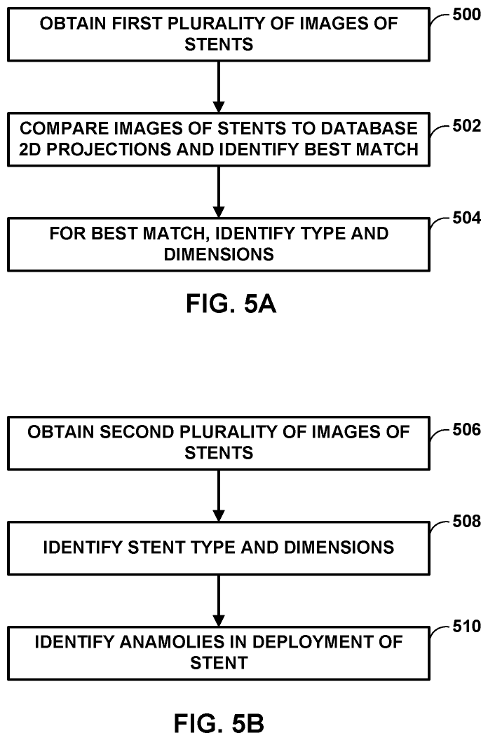

FIGS. 5A and 5B are flow diagrams illustrating example machine learning model training techniques according to one or more aspects of this disclosure. The techniques of FIGS. 5A and 5B are described below with respect to processing circuitry 204, but such techniques may be performed by any of, or any combination of, processing circuitry of devices depicted in FIG. 1 or capable of performing such techniques.

In some examples, processing circuitry 204 may train machine learning model(s) 222 by employing the techniques of both FIG. 5A and FIG. 5B. For example, processing circuitry 204 may train machine learning model(s) 222 to learn from images of properly deployed stents so the algorithm can learn about stent type, length, and diameter. Processing circuitry 204 may then train machine learning model(s) 222 with cases where stents are deployed sub-optimally, where a set of test images are provided with the specific information to be learned. For example, a person may manually input notes regarding the stent prior to performing a procedure.

Processing circuitry 204 may obtain a first plurality of images of stents (500). The first plurality of images of stents may include images of relatively ideal implantation locations, image quality, and/or implanted medical device condition. Processing circuitry 204 may compare images of stents to 2D projections in database 220 and identify a best match for each implanted medical device represented in the images (502). Processing circuitry 204 may, for each best match, identify the type and the dimensions of the implanted medical device (504). For example, processing circuitry 204 may utilize database 220 to identify the implanted medical device.

Processing circuitry 204 may obtain a second plurality of images of stents (506). The second plurality of images of stents may include images of relatively non-ideal implantation locations, image quality, and/or implanted medical device condition. Processing circuitry 204 may identify a stent type and dimensions (508). For example, processing circuitry 204 may compare images of stents to database 2D projections and identify a best match for each implanted device and utilize database 220 to identify the type and dimensions of the implanted medical device. Processing circuitry 204 may also identify anomalies in the deployment of the stent (510).

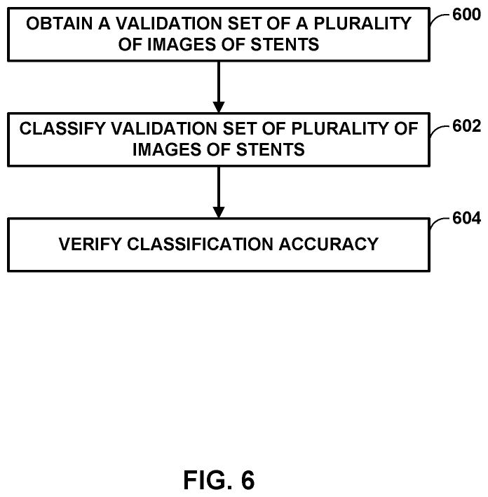

FIG. 6 is a flow diagram illustrating example machine learning model verification techniques according to one or more aspects of this disclosure. The techniques of FIG. 6 is described below with respect to processing circuitry 204, but such techniques may be performed by any of, or any combination of, processing circuitry of devices depicted in FIG. 1 or capable of performing such techniques. These validation images are provided without information and the algorithm presents output based on what it has learned. The accuracy of the algorithm is evaluated and if insufficient, additional training is performed.

Processing circuitry 204 may obtain a validation set of a plurality of images of stents (600). For example, these validation images are provided without any additional information to test the classifications provided by machine learning model(s) 222. information and the algorithm presents output based on what it has learned. The accuracy of the algorithm is evaluated and if insufficient, additional training is performed.

Processing circuitry 204 may classify the validation set of plurality of images of stents (602). For example, processing circuitry 204 may classify each image of the validation set as being an image of a particular respective stent. A person may verify the classification accuracy (604). For example, a person may review information associated with the images and/or the images themselves to determine if a given classification by processing circuitry 204 is correct. In some examples, the person may use supervised training to update machine learning model(s) 222, for example, based on the validation set and/or based on further training data, such as the first or second plurality of images (FIGS. 5A and 5B).

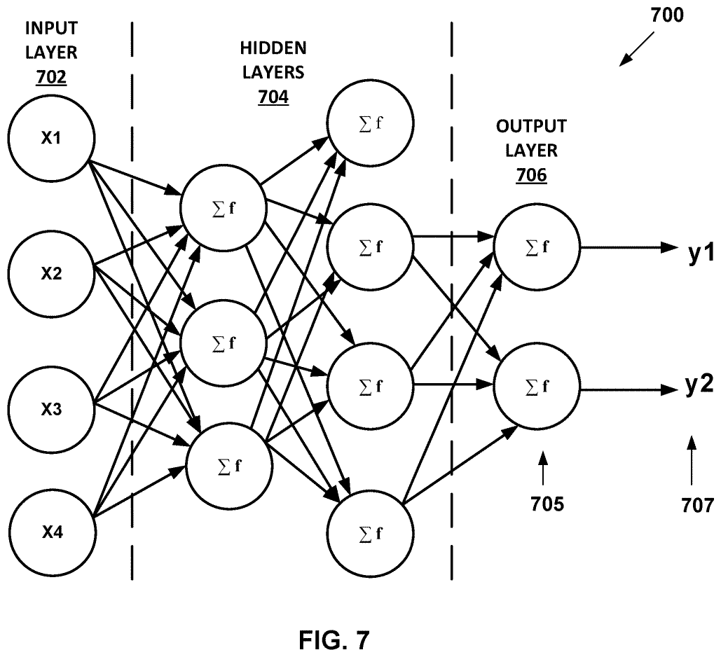

FIG. 7 is a conceptual diagram illustrating an example machine learning model according to one or more aspects of this disclosure. Machine learning model 700 may be an example of machine learning model(s) 222. Machine learning model 700 may be an example of a deep learning model, or deep learning algorithm, trained to determine an identification of an implanted medical device and/or to determine a recommended treatment strategy. One or more of computing device 150 and/or server 160 may train, store, and/or utilize machine learning model 700, but other devices of system 100 may apply inputs to machine learning model 700 in some examples. In some examples, other types of machine learning and deep learning models or algorithms may be utilized in other examples. For examples, a convolutional neural network model of ResNet-18 may be used. Some non-limiting examples of models that may be used for transfer learning include AlexNet, VGGNet, GoogleNet, ResNet50, or DenseNet, etc. Some non-limiting examples of machine learning techniques include Support Vector Machines, K-Nearest Neighbor algorithm, and Multi-layer Perceptron.

As shown in the example of FIG. 7, machine learning model 700 may include three types of layers. These three types of layers include input layer 702, hidden layers 704, and output layer 706. Output layer 706 comprises the output from the transfer function 705 of output layer 706. Input layer 702 represents each of the input values X1 through X4 provided to machine learning model 700. In some examples, the input values may include any of the values input into the machine learning model, as described above. For example, the input values may include image data 214 and/or 3D image model 216, as described above. In addition, in some examples input values of machine learning model 700 may include additional data, such as other data that may be collected by or stored in system 100.

Each of the input values for each node in the input layer 702 is provided to each node of a first layer of hidden layers 704. In the example of FIG. 7, hidden layers 704 include two layers, one layer having four nodes and the other layer having three nodes, but fewer or greater number of nodes may be used in other examples. Each input from input layer 702 is multiplied by a weight and then summed at each node of hidden layers 704. During training of machine learning model 700, the weights for each input are adjusted to establish the relationship between image data 214 and/or 3D image model 216 and an identification of a medical device and/or a recommended treatment strategy. In some examples, one hidden layer may be incorporated into machine learning model 700, or three or more hidden layers may be incorporated into machine learning model 700, where each layer includes the same or different number of nodes.

The result of each node within hidden layers 704 is applied to the transfer function of output layer 706. The transfer function may be linear or non-linear, depending on the number of layers within machine learning model 700. Example non-linear transfer functions may be a sigmoid function or a rectifier function. The output 707 of the transfer function may be a classification that image data 214 and/or 3D image model 216 is indicative of a particular implanted medical device and/or a particular recommended treatment strategy.

As shown in the example above, by applying machine learning model 700 to input data such as image data 214 and/or 3D image model 216, processing circuitry 204 is able to determine a particular implanted medical device and/or a particular recommended treatment strategy. This may improve the effectiveness of a medical procedure, the time a medical procedure takes to complete, improve patient outcomes, or the like.

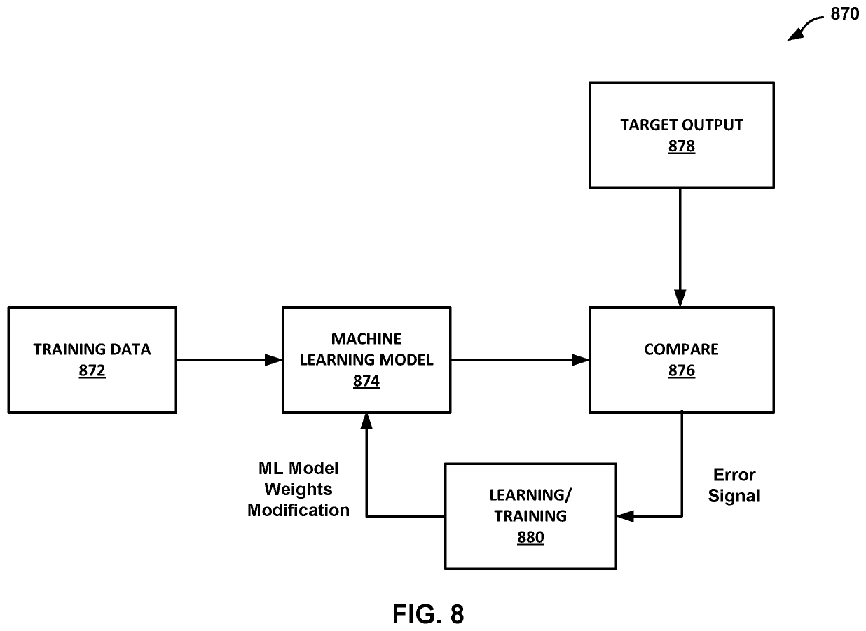

FIG. 8 is a conceptual diagram illustrating an example training process for a machine learning model according to one or more aspects of this disclosure. Process 870 may be used to train machine learning model(s) 222 or machine learning model 700. A machine learning model 874 (which may be an example of machine learning model 700 and/or machine learning model(s) 222) may be implemented using any number of models for supervised and/or reinforcement learning, such as but not limited to, an artificial neural network, a decision tree, naïve Bayes network, support vector machine, or k-nearest neighbor model, CNN, RNN, LSTM, ensemble network, to name only a few examples. In some examples, one or more of computing device 150 and/or server 160 initially trains machine learning model 874 based on a corpus of training data 872. Training data 872 may include, for example, “clean” images of anatomy without previous intervention and images of anatomy with previous intervention. In some examples, the images of anatomy with previous intervention may include a first plurality of images of relatively ideal implantation locations, image quality, and/or implanted medical device condition, a second plurality of images of relatively non-ideal implantation locations, image quality, and/or implanted medical device condition, and/or a plurality of verification images, as discussed above with respect to FIGS. 5A-6. Training data 872 may include data from past medical procedures performed on a plurality of patients having different patient conditions, different implanted medical devices, different prior medical procedures, tags, other training data mentioned herein, and/or the like. In some examples, training data 872 may include image data from past medical procedures performed relatively well, so as to train machine learning model 874 to provide treatment recommendations that are relatively better than average or are “expert” level recommendations. In some examples, processing circuitry 204 may execute machine learning model(s) 222 to generate 3D image model 216. In such examples, machine learning model(s) 222 may be trained using image data of “clean” anatomy (e.g., anatomy without previous intervention) and examples of image data of anatomy previously treated (e.g., anatomy with previous intervention).

While training machine learning model 874, processing circuitry of system 2 may compare 876 a prediction or classification with a target output 878. Processing circuitry 204 may utilize an error signal from the comparison to train (learning/training 880) machine learning model 874. Processing circuitry 204 may generate machine learning model weights or other modifications which processing circuitry 204 may use to modify machine learning model 874. For examples, processing circuitry 204 may modify the weights of machine learning model 874 based on the learning/training 480. For example, one or more of computing device 150 and/or server 160, may, for each training instance in training data 872, modify, based on training data 872, the manner in which a particular implanted medical device and/or a particular recommended treatment strategy is determined.

The techniques described in this disclosure may be implemented, at least in part, in hardware, software, firmware or any combination thereof. For example, various aspects of the described techniques may be implemented within one or more processors or processing circuitry, including one or more microprocessors, digital signal processors (DSPs), application specific integrated circuits (ASICs), field programmable gate arrays (FPGAs), or any other equivalent integrated or discrete logic circuitry, as well as any combinations of such components. The terms “controller”, “processor”, or “processing circuitry” may generally refer to any of the foregoing logic circuitry, alone or in combination with other logic circuitry, or any other equivalent circuitry. A control unit comprising hardware may also perform one or more of the techniques of this disclosure. Such hardware, software, and firmware may be implemented within the same device or within separate devices to support the various operations and functions described in this disclosure. In addition, any of the described units, circuits or components may be implemented together or separately as discrete but interoperable logic devices. Depiction of different features as circuits or units is intended to highlight different functional aspects and does not necessarily imply that such circuits or units must be realized by separate hardware or software components. Rather, functionality associated with one or more circuits or units may be performed by separate hardware or software components or integrated within common or separate hardware or software components.

The techniques described in this disclosure may also be embodied or encoded in a computer-readable medium, such as a computer-readable storage medium, containing instructions. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), or electronically erasable programmable read only memory (EEPROM), or other computer readable media.

This disclosure includes the following non-limiting examples.

Example 1. A medical system comprising: memory configured to store a 3-dimensional (3D) image of cardiac anatomy of a patient; and processing circuitry communicatively coupled to the memory, the processing circuitry being configured to: obtain image data of the cardiac anatomy of the patient; generate the 3D image of the cardiac anatomy of the patient based on the image data; determine an identification of an implanted medical device based on the 3D image; determine a recommended treatment strategy based on the 3D image and the identification of the implanted medical device; and output the recommended treatment strategy.

Example 2. The medical system of example 1, wherein as part of at least one of generating the 3D image of the cardiac anatomy of the patient based on the image data, determining the identification of the implanted medical device based on the 3D image, or determining the recommended treatment strategy based on the 3D image and the identification of the implanted medical device, the processing circuitry is configured to execute at least one machine learning model.

Example 3. The medical system of example 1 or example 2, wherein as part of determining the identification of the implanted medical device, the processing circuitry is configured to determine that at least one characteristic of the implanted medical device matches at least one characteristic of a stent in a stent database.

Example 4. The medical system of example 3, wherein the at least one characteristic comprises one or more of a type, a length, or a diameter.

Example 5. The medical system of any of examples 1-4, wherein the processing circuitry is further configured to determine an effectiveness of a previous medical procedure based on the 3D image and the identification of the implanted medical device.

Example 6. The medical system of any of examples 1-5, wherein the implanted medical device comprises a sternal tie.

Example 7. The medical system of any of examples 1-6, wherein as part of determining the recommended treatment strategy, the processing circuitry is configured to determine a path to an artery of the cardiac anatomy having a chronic total occlusion and a diameter of the artery of the cardiac anatomy having the chronic total occlusion and wherein the recommended treatment strategy comprises the path to the artery and the diameter of the artery.

Example 8. The medical system of example 7, wherein the recommended treatment strategy comprises a recommended retrograde path and at least one recommended equipment.

Example 9. The medical system of any of examples 1-8, wherein the image data comprises at least one of fluoroscopy data, intravascular ultrasound (IVUS) data, or optical coherence tomography (OCT) fractional flow reserve (FFR) data.

Example 10. The medical system of any of examples 1-9, wherein the processing circuitry is further configured to obtain a database of implantable medical devices.

Example 11. The medical system of example 10, wherein the processing circuitry is configured to generate the database of implantable medical devices, wherein as part of generating the database of implantable medical devices, the processing circuitry is configured to: create a respective 3D models of each of a plurality of implantable medical devices; create a plurality of 2D projections of each respective 3D model; collate lengths and diameters of the plurality of 2D projections of each respective 3D model; and define rules for the database of implantable medical devices.

Example 12. The medical system of any of examples 1-11, wherein the machine learning model is trained on a plurality of fluoroscopy images of implanted medical devices.

Example 13. The medical system of example 12, wherein the plurality of fluoroscopy images comprises a first set of fluoroscopy images of implanted medical devices and a second set of fluoroscopy images of implanted medical devices, wherein the second set of fluoroscopy images comprise images of implanted medical devices in less desirable implantation situations than the first set of fluoroscopy images.

Example 14. The medical system of example 13, wherein the training of the machine learning model is verified using a third set of fluoroscopy images of implanted medical devices.

Example 15. A method comprising: obtaining, by a medical system, image data of cardiac anatomy of a patient; generating, by the medical system, a 3D image of the cardiac anatomy of the patient based on the image data; determining an identification of an implanted medical device based on the 3D image; determining a recommended treatment strategy based on the 3D image and the identification of the implanted medical device; and outputting the recommended treatment strategy.

Example 16. The method of example 15, wherein at least one of generating the 3D image of the cardiac anatomy of the patient based on the image data, determining the identification of the implanted medical device based on the 3D image, or determining the recommended treatment strategy based on the 3D image and the identification of the implanted medical device, comprises executing at least one machine learning model.

Example 17. The method of example 15 or example 16, wherein determining the identification of the implanted medical device comprises determining that at least one characteristic of the implanted medical device matches at least one characteristic of a stent in a stent database.

Example 18. The method of example 17, wherein the at least one characteristic comprises one or more of a type, a length, or a diameter.

Example 19. The method of any of examples 15-18, further comprising determining an effectiveness of a previous medical procedure based on the 3D image and the identification of the implanted medical device.

Example 20. A non-transitory computer-readable storage medium storing instructions, which when executed cause processing circuitry to: obtain image data of cardiac anatomy of a patient; generate a 3D image of the cardiac anatomy of the patient based on the image data; determine an identification of an implanted medical device based on the 3D image; determine a recommended treatment strategy based on the 3D image and the identification of the implanted medical device; and output the recommended treatment strategy.

Various examples have been described. These and other examples are within the scope of the following claims.

1. A medical system comprising:
memory configured to store a 3-dimensional (3D) image of cardiac anatomy of a patient; and
processing circuitry communicatively coupled to the memory, the processing circuitry being configured to:
obtain image data of the cardiac anatomy of the patient;
generate the 3D image of the cardiac anatomy of the patient based on the image data;
determine an identification of an implanted medical device based on the 3D image;
determine a recommended treatment strategy based on the 3D image and the identification of the implanted medical device; and
output the recommended treatment strategy.

2. The medical system of claim 1, wherein as part of at least one of generating the 3D image of the cardiac anatomy of the patient based on the image data, determining the identification of the implanted medical device based on the 3D image, or determining the recommended treatment strategy based on the 3D image and the identification of the implanted medical device, the processing circuitry is configured to execute at least one machine learning model.

3. The medical system of claim 1, wherein as part of determining the identification of the implanted medical device, the processing circuitry is configured to determine that at least one characteristic of the implanted medical device matches at least one characteristic of a stent in a stent database.

4. The medical system of claim 3, wherein the at least one characteristic comprises one or more of a type, a length, or a diameter.

5. The medical system of claim 1, wherein the processing circuitry is further configured to determine an effectiveness of a previous medical procedure based on the 3D image and the identification of the implanted medical device.

6. The medical system of claim 1, wherein the implanted medical device comprises a sternal tie.

7. The medical system of claim 1, wherein as part of determining the recommended treatment strategy, the processing circuitry is configured to determine a path to an artery of the cardiac anatomy having a chronic total occlusion and a diameter of the artery of the cardiac anatomy having the chronic total occlusion and wherein the recommended treatment strategy comprises the path to the artery and the diameter of the artery.

8. The medical system of claim 7, wherein the recommended treatment strategy comprises a recommended retrograde path and at least one recommended equipment.

9. The medical system of claim 1, wherein the image data comprises at least one of fluoroscopy data, intravascular ultrasound (IVUS) data, or optical coherence tomography (OCT) fractional flow reserve (FFR) data.

10. The medical system of claim 1, wherein the processing circuitry is further configured to obtain a database of implantable medical devices.

11. The medical system of claim 10, wherein the processing circuitry is configured to generate the database of implantable medical devices, wherein as part of generating the database of implantable medical devices, the processing circuitry is configured to:
create a respective 3D model of each of a plurality of implantable medical devices;
create a plurality of 2D projections of each respective 3D model;
collate lengths and diameters of the plurality of 2D projections of each respective 3D model; and
define rules for the database of implantable medical devices.

12. The medical system of claim 2, wherein the machine learning model is trained on a plurality of fluoroscopy images of implanted medical devices.

13. The medical system of claim 12, wherein the plurality of fluoroscopy images comprises a first set of fluoroscopy images of implanted medical devices and a second set of fluoroscopy images of implanted medical devices, wherein the second set of fluoroscopy images comprise images of implanted medical devices in less desirable implantation situations than the first set of fluoroscopy images.

14. A method comprising:
obtaining, by a medical system, image data of cardiac anatomy of a patient;
generating, by the medical system, a 3D image of the cardiac anatomy of the patient based on the image data;
determining an identification of an implanted medical device based on the 3D image;
determining a recommended treatment strategy based on the 3D image and the identification of the implanted medical device; and
outputting the recommended treatment strategy.

15. A non-transitory computer-readable storage medium storing instructions, which when executed cause processing circuitry to:
obtain image data of cardiac anatomy of a patient;
generate a 3D image of the cardiac anatomy of the patient based on the image data;
determine an identification of an implanted medical device based on the 3D image;
determine a recommended treatment strategy based on the 3D image and the identification of the implanted medical device; and
output the recommended treatment strategy.

16. The medical system of claim 13, wherein the training of the machine learning model is verified using a third set of fluoroscopy images of implanted medical devices.

17. The method of claim 14, wherein at least one of generating the 3D image of the cardiac anatomy of the patient based on the image data, determining the identification of the implanted medical device based on the 3D image, or determining the recommended treatment strategy based on the 3D image and the identification of the implanted medical device, comprises executing at least one machine learning model.

18. The method of claim 14, wherein determining the identification of the implanted medical device comprises determining that at least one characteristic of the implanted medical device matches at least one characteristic of a stent in a stent database.

19. The method of claim 17, wherein the at least one characteristic comprises one or more of a type, a length, or a diameter.

20. The method of claim 14, further comprising determining an effectiveness of a previous medical procedure based on the 3D image and the identification of the implanted medical device.
